SEQUENCE LISTING (19 sequences)

SEQ ID NO: 1 - DNA, 7499 bp, Chlamydia trachomatis
ggtaagtcctctagtacaaacacccccaatattgtgatataattaaaattatattcatattctgttgccagaaaaaacacttttaggctatattagagccatcttctttgaagcgttgtcttctcgagaagatttatcgtacgcaaatatcatctttgcggttgcgtgtcctgtgaccttcattatgtcggagtctgagcaccctaggcgtttgtactccgtcacagcggttgctcgaagcacgtgcggggttatcttaaaagggattgcagcttgtagtcctgcttgagagaacgtgcgggcgatttgccttaaccccaccatttttccggagcgagttacgaagacaaaacctcttcgttgaccgatgtactcttgtagaaagtgcataaacttctgaggataagttataataatcctcttttctgtctgacggttcttaagctgggagaaagaaatggtagcttgttggaaacaaatctgactaatctccaagcttaagacttcagaggagcgtttacctccttggagcattgtctgggcgatcaaccaatcccgggcattgattttttttagctcttttaggaaggacgctgtttgcaaactgttcatcgcatctgtttttactatttccctggttttaaaaaatgttcgactattttcttgtttagaaggttgcgctatagcgactattccttgagtcatcctgtttaggaatcttgttaaggaaatatagcttgctgctcgaacttgtttagtaccttcggtccaagaagtcttggcagaggaaacttttttaatcgcatctagaattagattatgatttaaaagggaaaactcttgcagattcatatccaaggacaatagaccaatcttttctaaagacaaaaaagatcctcgatatgatctacaagtatgtttgttgagtgatgcggtccaatgcataataacttcgaataaggagaagcttttcatgcgtttccaataggattcttggcgaatttttaaaacttcctgataagacttttcgctatattctaacgacatttcttgctgcaaagataaaatccctttacccatgaaatccctcgtgatataacctatccgtaaaatgtcctgattagtgaaataatcaggttgttaacaggatagcacgctcggtatttttttatataaacatgaaaactcgttccgaaatagaaaatcgcatgcaagatatcgagtatgcgttgttaggtaaagctctgatatttgaagactctactgagtatattctgaggcagcttgctaattatgagtttaagtgttctcatcataaaaacatattcatagtatttaaatacttaaaagacaatggattacctataactgtagactcggcttgggaagagcttttgcggcgtcgtatcaaagatatggacaaatcgtatctcgggttaatgttgcatgatgctttatcaaatgacaagcttagatccgtttctcatacggttttcctcgatgatttgagcgtgtgtagcgctgaagaaaatttgagtaatttcattttccgctcgtttaatgagtacaatgaaaatccattgcgtagatctccgtttctattgcttgagcgtataaagggaaggcttgacagtgctatagcaaagactttttctattcgcagcgctagaggccggtctatttatgatatattctcacagtcagaaattggagtgctggctcgtataaaaaaaagacgagcaacgttctctgagaatcaaaattctttctttgatgccttcccaacaggatacaaggatattgatgataaaggagttatcttagctaaaggtaatttcgtgattatagcagctaggccatctatagggaaaactgctttagctatagacatggcgataaatcttgcggttactcaacagcgtagagttggtttcctatctctagaaatgagcgcaggtcaaattgttgagcggattattgctaatttaacaggaatatctggtgaaaaattacaaagaggggatctctctaaagaagaattattccgagtagaagaagctggagaaacagttagagaatcacatttttatatctgcagtgatagtcagtataagcttaatttaatcgcgaatcagatccggttgctgagaaaagaagatcgagtagacgtaatatttatcgattacttgcagttgatcaactcatcggttggagaaaatcgtcaaaatgaaatagcagatatatctagaaccttaagaggtttagcctcagagctaaacattcctatagtttgtttatcccaactatctagaaaagttgaggatagagcaaataaagttcccatgctttcagatttgcgagacagcggtcaaatagagcaagacgcagatgtgattttgtttatcaataggaaggaatcgtcttctaattgtgagataactgttgggaaaaatagacatggatcggttttctcttcggtattacatttcgatccaaaaattagtaaattctccgctattaaaaaagtatggtaaattatagtaactgccacttcatcaaaagtcctatccaccttgaaaatcagaagtttggaagaagacctggtcaatctattaagatatctcccaaattggctcaaaatgggatggtagaagttataggtcttgattttctttcatctcattaccatgcattagcagctatccaaagattgctgactgcaacgaattacaaggggaacacaaaaggggttgttttatccagagaatcaaatagttttcaatttgaaggatggataccaagaatccgttttacaaaaactgaattcttagaggcttatggagttaagcggtataaaacatccagaaataagtatgagtttagtggaaaagaagctgaaactgctttagaagccttataccatttaggacatcaaccgtttttaatagtggcaactagaactcgatggactaatggaacacaaatagtagaccgttaccaaactctttctccgatcattaggatttacgaaggatgggaaggtttaactgacgaagaaaatatagatatagacttaacaccttttaattcaccatctacacggaaacataaagggttcgttgtagagccatgtcctatcttggtagatcaaatagaatcctactttgtaatcaagcctgcaaatgtataccaagaaataaaaatgcgcttcccaaatgcatcaaagtatgcttacacatttatcgactgggtgattacagcagctgcgaaaaagagacgaaaattaactaaggataattcttggccagaaaacttgttcttaaacgttaacgttaaaagtcttgcatatattttaaggatgaatcggtacatttgtacaaggaactggaaaaaaatcgagttagctatcgataaatgtatagaaatcgccattcagcttggttggttatctagaagaaaacgcattgaatttctggattcttctaaactctctaaaaaagaaattctatatctaaataaagagcgttttgaagaaataactaagaaatctaaagaacaaatggaacaattagaacaagaatctattaattaatagcaaacttgaaactaaaaacctaatttatttaaagctcaaaataaaaaagagttttaaaatgggaaattctggtttttatttgtataacactcaaaactgcgtctttgctgataatatcaaagttgggcaaatgacagagccgctcaaggaccagcaaataatccttgggacaacatcaacacctgtcgcagccaaaatgacagcttctgatggaatatctttaacagtctccaataatccatcaaccaatgcttctattacaattggtttggatgcggaaaaagcttaccagcttattctagaaaagttgggagatcaaattcttggtggaattgctgatactattgttgatagtacagtccaagatattttagacaaaatcacaacagacccttctctaggtttgttgaaagcttttaacaactttccaatcactaataaaattcaatgcaacgggttattcactcccaggaacattgaaactttattaggaggaactgaaataggaaaattcacagtcacacccaaaagctctgggagcatgttcttagtctcagcagatattattgcatcaagaatggaaggcggcgttgttctagctttggtacgagaaggtgattctaagccctacgcgattagttatggatactcatcaggcgttcctaatttatgtagtctaagaaccagaattattaatacaggattgactccgacaacgtattcattacgtgtaggcggtttagaaagcggtgtggtatgggttaatgccctttctaatggcaatgatattttaggaataacaaatacttctaatgtatcttttttggaggtaatacctcaaacaaacgcttaaacaatttttattggatttttcttataggttttatatttagagaaaaaagttcgaattacggggtttgttatgcaaaataaaagcaaagtgagggacgattttattaaaattgttaaagatgtgaaaaaagatttccccgaattagacctaaaaatacgagtaaacaaggaaaaagtaactttcttaaattctcccttagaactctaccataaaagtgtctcactaattctaggactgcttcaacaaatagaaaactctttaggattattcccagactctcctgttcttgaaaaattagaggataacagtttaaagctaaaaaaggctttgattatgcttatcttgtctagaaaagacatgttttccaaggctgaatagataacttactctaacgttggagttgatttgcacaccttagttttttgctcttttaagggaggaactggaaaaacaacactttctctaaacgtgggatgcaacttggcccaatttttagggaaaaaagtgttacttgctgacctagacccgcaatccaatttatcttctggattgggggctagtgtcagaagtaaccaaaaaggcttacacgacatagtatacacatcaaacgatttaaaatcaatcatttgcgaaacaaaaaaagatagtgtggacctaattcctgcatcatttttatccgaacagtttagagaattggatattcatagaggacctagtaacaacttaaagttatttctgaatgagtactgcgctcctttttatgacatctgcataatagacactccacctagcctaggagggttaacgaaagaagcttttgttgcaggagacaaattaattgcttgtttaactccagaacctttttctattctagggttacaaaagatacgtgaattcttaagttcggtcggaaaacctgaagaagaacacattcttggaatagctttgtctttttgggatgatcgtaactcgactaaccaaatgtatatagacattatcgagtctatttacaaaaacaagcttttttcaacaaaaattcgtcgagatatttctctcagccgttctcttcttaaagaagattctgtagctaatgtctatccaaattctagggccgcagaagatattctgaagttaacgcatgaaatagcaaatattttgcatatcgaatatgaacgagattactctcagaggacaacgtgaacaaactaaaaaaagaagcgaatgtcttttttaaaaaaaatcaaactgccgcttctttagattttaagaagacgcttccttccattgaactattctcagcaactttgaattctgaggaaagtcagagtttggatcaattatttttatcagagtcccaaaactattcggatgaagaattttatcaagaagacatcctagcggtaaaactgcttactggtcagataaaatccatacagaagcaacacgtacttcttttaggagaaaaaatctataatgctagaaaaatcctgagtaaggatcacttctcctcaacaactttttcatcttggatagagttagtttttagaactaagtcttctgcttacaatgctcttgcatattacgagctttttataaacctccccaaccaaactctacaaaaagagtttcaatcgatcccctataaatccgcatatattttggccgctagaaaaggcgatttaaaaaccaaggtcgatgtgatagggaaagtatgtggaatgtcgaactcatcggcgataagggtgttggatcaatttcttccttcatctagaaacaaagacgttagagaaacgatagataagtctgattcagagaagaatcgccaattatctgatttcttaatagagatacttcgcatcatgtgttccggagtttctttgtcctcctataacgaaaatcttctacaacagctttttgaactttttaagcaaaagagctgatcctccgtcagctcatatatatatctattatatatatatatttagggatttgattttacgagagagatttgcaactcttggtggtagactttgcaactcttggtggtagactttgcaactcttggtggtagactttgcaactcttggtggtagacttggtcataatggacttttgttgaaaaatttcttaaaatcttagagctccgattttgaatagctttggttaagaaaatgggctcgatggctttccataaaagtaggttgttcttaacttttggggacgcgtcggaaatttggttatctactttatctcatctaactagaaaaaattatgcgtctgggattaactttcttgtttctttagagattctggatttatcggaaaccttgataaaggctatttctcttgaccacagcgaatctttgtttaaaatcaagtctctagatgtttttaatggaaaagtcgtttcagaggcctctaaacaggctagagcggcatgctacatatctttcacaaagtttttgtatagattgaccaagggatatattaaacccgctattccattgaaagattttggaaacactacattttttaaaatccgagacaaaatcaaaacagaatcgatttctaagcaggaatggacagttttttttgaagcgctccggatagtgaattatagagactatttaatcggtaaattgattgtacaagggatccgtaagttagacgaaattttgtctttgcgcacagacgatctattttttgcatccaatcagatttcctttcgcattaaaaaaagacagaataaagaaaccaaaattctaatcacatttcctatcagcttaatggaggagttgcaaaaatacacttgtgggagaaatgggagagtatttgtttctaaaatagggattcctgtaacaacaagtcaggttgcgcataattttaggcttgcagagttctatagtgctatgaaaaaaaaattactcctagagtacttcgtgcaagcgctttgattcatttaaagcaaataggattaaaagatgaggaaatcatgcgtatttcctgtctttcatcgagacaaagtgtgtgttcttattgttctggggaaga

SEQ ID NO: 2 - DNA, 23 bp, Chlamydia trachomatis
ggattgactccgacaacgtattc

SEQ ID NO: 3 - DNA, 22 bp, Chlamydia trachomatis
tgccctttctaatggcaatgat

SEQ ID NO: 4 - DNA, 23 bp, Chlamydia trachomatis
ggattgactccgacaacgtattc

SEQ ID NO: 5 - DNA, 22 bp, Chlamydia trachomatis
atcattgccattagaaagggca

SEQ ID NO: 6 - DNA, 640 bp, Neisseria gonorrhoeae
tcagacgtatagcggattaacaaaaatcaggacaaggggcgggccgcaggcagtacggatggtacggaaccgatccgcccgtgcttcagcaccttagggaaccgttccctttgagccggggcggggcaacgccgtaccggtttttgttcatccgccatattgtgttgaaacaccgcccggaacccgatataatccgcccttcaacatcagtgaaaatctttttttaaccggtcaaaccgaataaggagccgaaaatgaatccagcccgcaaaaaaccttctcttctcttctcttctcttctcttctcttctcttccgcagcgcaggcggcaagtgaaggcaatggccgcggcccgtatgtgcaggcggatttagcctacgccgccgaacgcattacccacgattatccggaaccaaccggtgcaaaaaaaggcacaacaataagcacggtaagcgattatttcagaaacatccgtacgcattccatccacccccgggtgtcggtcggctacgacttcggcggctggaggatagcggcagattatgcccgttacagaaagtggaacaacaataaatattccgtgagcataaaagagttgctaagaaacaagggcaatggcaacaggacagacctgaaggcg

SEQ ID NO: 7 - DNA, 17 bp, Neisseria gonorrhoeae
gttgaaacaccgcccgg

SEQ ID NO: 8 - DNA, 24 bp, Neisseria gonorrhoeae
atctttttttaaccggtcaaaccg

SEQ ID NO: 9 - DNA, 17 bp, Neisseria gonorrhoeae
gttgaaacaccgcccgg

SEQ ID NO: 10 - DNA, 24 bp, Neisseria gonorrhoeae
cggtttgaccggttaaaaaaagat

SEQ ID NO: 11 - DNA, 35 bp, Chlamydia trachomatis
tcggtttgaccggttaaaaaaagattttcactgat

SEQ ID NO: 12 - DNA, 34 bp, Neisseria gonorrhoeae
ggattgactccracaacgtattcattacgtgtag

SEQ ID NO: 13 - DNA, 132 bp, Artificial Sequence
  Control DNA
ggattgactccgacaacgtattcccgtaaaatccagcacgtaagccgatacatagaatgcacatccgccaaatctttcgccagaatcgcaatagcggatctcctgcagcaccttcccgggcggtgtttcaac

SEQ ID NO: 14 - DNA, 19 bp, Neisseria gonorrhoeae
cccttcaacatcagtgaaa

SEQ ID NO: 15 - DNA, 19 bp, Neisseria gonorrhoeae
ctttgaaccatcagtgaaa

SEQ ID NO: 16 - DNA, 15 bp, Neisseria gonorrhoeae
acccgatataatccg

SEQ ID NO: 17 - DNA, 29 bp, Chlamydia trachomatis
acaccgctttctaaaccgcctacacgtaa

SEQ ID NO: 18 - DNA, 29 bp, Artificial Sequence
  probe for control DNA
tctggcgaaagatttggcggatgtgcatt

SEQ ID NO: 19 - DNA, 130 bp, Artificial Sequence
  Control DNA
gttgaacaccgcccggccgtaaaatccagcacgtaagccgatacatagaatgcacatccgccaaatctttcgccagaatcgcaatagcggatctcctgcagcaccttctgccctttctaatggcaatgat

(19) United States
(12) Patent Application Publication
(10) Pub. No.: US 2013/0045476 A1
(43) Pub. Date: Feb. 21, 2013

(54) METHOD FOR COMBINED MONITORING OF DETECTION OF AT LEAST TWO MOLECULAR TARGETS AND TO A KIT THEREFOR

(71) Applicants: Paul Hendrik Maria Savelkoul, Amsterdam (NL); Maria Hendrika Anna Hemans, Heesbeen (NL)

(72) Inventors: Paul Hendrik Maria Savelkoul, Amsterdam (NL); Maria Hendrika Anna Hemans, Heesbeen (NL)

(21) Appl. No.: 13/578,543

(22) PCT Filed: Feb. 11, 2011
(86) PCT No.: PCT/NL2011/050097

§ 371 Date: Oct. 22, 2012

Related U.S. Application Data
(60) Provisional application No. 61/304,082, filed on Feb. 12, 2010.

(51) Int. Cl. C12Q 1/68; C12M 1/34; C12N 1/21; C07H 21/04; C12N 15/74

(52) U.S. Cl. 435/6.11; 435/6.12; 536/23.1; 435/320.1; 435/252.3; 435/287.2

(57) ABSTRACT

The invention relates to methods for combined monitoring of detection of at least two molecular targets.

### CROSS-REFERENCE TO RELATED APPLICATIONS

This application is a national phase entry under 35 U.S.C. §371 of International Patent Application PCT/NL2011/050097, filed Feb. 11, 2011, designating the United States of America and published in English as International Patent Publication WO 2011/099855 A1 on Aug. 18, 2011, which claims the benefit under Article 8 of the Patent Cooperation Treaty and under 35 U.S.C. §119(e) to U.S. Provisional Patent Application Ser. No. 61/304,082, filed Feb. 12, 2010.

### TECHNICAL FIELD

The present invention relates to a method for combined monitoring of detection of at least two molecular targets and to a kit therefor.

### BACKGROUND

Chlamydia trachomatis is a species of the chlamydiae, a group of intracellular bacteria. It causes sexually transmitted diseases, such as chlamydia and lymphogranuloma venereum, as well as trachoma, an eye infection that is a frequent cause of blindness. Neisseria gonorrhoeae is a species of gram-negative bacteria responsible for the disease gonorrhoea. Both infections are two known causes of ectopic pregnancy and can also lead to infertility if untreated. They are also known causes of the acute clinical syndromes of mucopurulent cervicitis and pelvic inflammatory disease. Co-infection of Neisseria gonorrhoeae and Chlamydia trachomatis is frequently observed. Therefore, the detection of Neisseria gonorrhoeae and Chlamydia trachomatis infections, which can be asymptomatic, especially in females, is of importance to individuals in need of treatment and to broader populations at risk of acquiring and further propagating the infections.

Traditional testing for the presence of Chlamydia trachomatis (CT) and/or Neisseria gonorrhoeae (NG) involves the culture of samples collected from patients, such as urethral specimen, on species-specific culture media. Such cultures are time-consuming and laborious. Testing methods based on nucleic acid amplification have, therefore, been developed.

For example, the FDA-approved AMPLICOR™ CT/NG kit available from Roche Diagnostic enables the combined detection of Chlamydia trachomatis or Neisseria gonorrhoeae. The method involves the amplification of Neisseria gonorrhoeae and Chlamydia trachomatis targets by amplification primers. The detection of the amplicons produced by these primers can be carried out, either through agarose gel techniques detecting amplified DNA directly, or by detecting amplified DNA through hybridization of a probe to amplified DNA, the bound probe can, for instance, be measured through colorimetric determination.

In enzyme-based amplification processes such as PCR, the efficiency of the reaction can be reduced by the presence of inhibitors that may be present in the sample. Therefore, in order to prevent the detection of false negative samples due to the presence of such inhibitors, the AMPLICOR™ CT/NG kit provides a CT/NG Internal Control to identify samples that may contain such inhibitors. With a “false negative,” a “false negative result,” or a “false negative sample,” according to the invention, is meant that in a particular test, wherein a sample comprises Chlamydia Trachomatis (CT) and/or Neisseria gonorrhoeae (NG), the test fails to detect CT and/or NG. The CT/NG Internal Control is a non-infectious recombinant plasmid DNA with primer-binding regions identical to those of the Chlamydia trachomatis target sequence, a randomized internal sequence of similar length and base composition as the NG and Chlamydia trachomatis target sequences, and a unique probe binding region distinct from the target amplicon. These features were selected to ensure equivalent amplification of the CT/NG Internal Control and CT/NG target DNA. The CT/NG Internal Control is introduced into each amplification reaction to be co-amplified with target DNA from the clinical specimen. When the CT/NG Internal Control is not amplified and thus not detected, it can be concluded that inhibitors are present, and the specimen may be alternatively tested with, for instance, a traditional culture test, thereby avoiding false negatives.

However, with the nucleic acid amplification methods and kits as described above, there still remains a risk for false negatives. For instance, the CT/NG Internal Control only controls for the amplification reaction, and not for the sample processing. Thus, in the case where during the processing of the sample the sample DNA is degraded or lost, the sample may become falsely negative for Chlamydia trachomatis and/or Neisseria gonorrhoeae, as the CT/NG Internal Control is added to the amplification reaction after the samples have been processed. Furthermore, as a relative large amount of the CT/NG Internal Control is used, and the CT/NG Internal Control is designed to ensure equivalent amplification, samples containing a very low amount of Chlamydia trachomatis run the risk of becoming false negative for Chlamydia trachomatis as the CT/NG Internal Control competes with the sample DNA for amplification. Finally, in some samples, either Chlamydia trachomatis or Neisseria gonorrhoeae could provide for such a strong positive signal that it becomes technically difficult, or impossible, to detect a possibly weaker signal for either Neisseria gonorrhoeae or Chlamydia trachomatis, respectively. In such instances, the AMPLICOR™ CT/NG method and kit does not allow for the detection of the possible weaker signal, thereby missing possible co-infections. Regarding the above, when using the AMPLICOR™ CT/NG method and kit, there is a chance that test results are falsely negative for Chlamydia trachomatis and/or Neisseria gonorrhoeae.

### DISCLOSURE

Provided are methods and kits for combined monitoring of detection of at least two molecular targets. The methods and kits may solve at least one of the problems with regard to the risk of possible false negative test results. The methods and kits as disclosed, may also solve other problems, which may become apparent from the description.

Provided is a method for combined monitoring of detection of at least two molecular targets, which method comprises providing a DNA sample, a control DNA, a primer pair for a first molecular target and a primer pair for a second molecular target and amplifying the DNA with the primer pairs, wherein the control DNA is capable of being amplified with one of the primers for the first molecular target and one of the primers for the second molecular target.

“Monitoring of detection,” according to the invention, is defined as detecting the presence, absence and/or amount of a molecular target. A molecular target according to the invention may be a DNA and/or RNA sequence of an organism. When reference is made to “monitoring of detection of an organism,” it is understood that from the organism, a DNA and/or RNA sequence, i.e., a molecular target, is monitored for detection. Thus, according to the invention, monitoring of detection of a molecular target, may result in detection of the presence, absence and/or amount of an organism.

Organisms, of which detection can be monitored, may include pathogenic microorganisms (e.g., fungi, yeast, bacteria, parasites), and may also include viruses, but is not necessarily limited thereto. Examples of organisms, from which DNA and/or RNA sequences, i.e., molecular targets, of which detection may be monitored in a method and kit according to the invention include Chlamydia Trachomatis, Neisseria gonorrhoeae, Mycobacterium tuberculosis, Trichomonas vaginalis (gram-positive and gram-negative bacteria), Candida spp (yeast), Aspergillus spp (fungus), Herpes viridae (virus), and Giardia lambia (parasite). Further examples include Actinomyces israelii, Bacteroides fragilis, Branhamella catarrhalis, Candida albicans, Candida glabrata, Candida krusei, Candida parapsilosis, Candida tropicalis, Citrobacter freundii, Clostridium perfringens, Cryptococcus neoformans, Cytomegalovirus, Enterobacter cloacae, Enterococcus faecalis, Enterococcus faecium, Epstein-Barr Virus, Escherichia coli, Gardnerella vaginalis, Haemophilus influenzae, Herpes simplex virus 1, Herpes simplex virus 2, Klebsiella pneumoniae, Lactobacillus species, Legionella pneumophila, Morganella morganii, Neisseria cinerea, Neisseria elongate, Neisseria flavescens, Neisseria lactamica, Neisseria meningitides, Neisseria mucosa, Neisseria perflava, Neisseria polysaccharea, Neisseria sicca, Neisseria subflava, Neisseria denitrificans, Peptostreptococcus species, Proteus mirabilis, Pseudomonas aeruginosa, Serratia marcescens, Staphylococcus aureus, Staphylococcus epidermidis, Streptococcus agalactiae, Streptococcus pneumoniae, Streptococcus pyogenes, Trichomonas vaginalis, and Yersinia enterocolitica.

The principles underlying the methods and kits according to the invention have been worked out below for the combined monitoring of detection of Chlamydia trachomatis and Neisseria gonorrhoeae. The principles for combined monitoring of detection of at least two molecular targets (organisms), or more, allows the skilled person, knowing the DNA and/or RNA sequences of the at least two organisms (molecular targets), to design a method and kit according to the invention with undue burden, as the principles as outlined below enables the skilled person to design such a method and/or kit according to the invention. Thus, it is to be understood that the method for the combined detection of Chlamydia trachomatis and Neisseria gonorrhoeae as described below does not limit the scope of the invention and or kit in any way.

In a preferred embodiment, the invention provides for a method for combined monitoring of detection of Chlamydia trachomatis and Neisseria gonorrhoeae, which method comprises providing a DNA sample, a control DNA, a primer pair for Chlamydia trachomatis and a primer pair for Neisseria gonorrhoeae, and amplifying the DNA with the primer pairs, wherein the control DNA is capable of being amplified with one of the primers for Chlamydia trachomatis and one of the primers for Neisseria gonorrhoeae.

### BRIEF DESCRIPTION OF THE DRAWING

FIG. 1. Schematic representation showing two molecular targets, for CT and NG, and control DNA (IAC) and forward and reverse primers as well as selectors and probes.

### DETAILED DESCRIPTION

The person skilled in the art will understand that with a “DNA sample” is meant a sample that is obtained from an organism that comprises DNA. Such an organism may be a person or a patient that may be at risk or suspected of being infected. For instance, a DNA sample may be obtained from a person at risk of having contracted Chlamydia trachomatis and/or Neisseria gonorrhoeae, or a patient having symptoms of a Chlamydia trachomatis and/or Neisseria gonorrhoeae infection. Such DNA samples may also be referred to as “clinical samples” or “clinical specimens.” The DNA sample used is not particularly subject to restrictions. Examples of DNA samples comprise urine, urethral (urinary tube) scrapings, cervical smears, anal smears, etc. The samples may, for instance, in particular be obtained from sites, e.g., the eye, urethra, cervix and/or anus, which are suspected of being infected by Chlamydia trachomatis and/or Neisseria gonorrhoeae.

Furthermore, a primer pair is provided for the first molecular target such as CT and a primer pair is provided for the second molecular target such as NG. Other primer pairs may also be optionally provided for further molecular targets, as the method of the invention may include monitoring of detection of further molecular targets (organisms) as well. The design of primer pairs, for example, for Chlamydia trachomatis and Neisseria gonorrhoeae, can be performed using several web-based applications available on the interne or using laboratory software. Sequence information for Chlamydia trachomatis and Neisseria gonorrhoeae is publicly available. Sequence variants may be observed that may have an effect on DNA amplification should such a variation have an effect on the DNA sequence of the primer and/or hybridization probe binding sites. Primers and/or probes may be designed taking into account such sequence variation (see Example 4). Chlamydia trachomatis is one of the non-gonococcal urethritis pathogens that contains a cryptic plasmid (M. Commanducci et al., Mol. Microbiol., 2, No. 4 (1998), pp. 531-538). Primer pairs are, therefore, preferably designed on the basis of this cryptic plasmid, but other sequences of Chlamydia trachomatis may also be used. The design of primers is preferably based on the less variable or invariable DNA sequences of Chlamydia trachomatis and/or Neisseria gonorrhoeae or focuses on a particular variant subset that would, for instance, be dominant in a particular (patient) population. In addition, sequence variation may be taking into account primer design. As long as the designed primer pairs are capable of hybridizing with their respective target DNA, i.e., Chlamydia trachomatis DNA or Neisseria gonorrhoeae DNA, and amplifying it in a PCR reaction, such primers are suitable for the invention.

According to the invention, the length of the primers for DNA amplification is, for example, between 10 and 40 nucleotides. The position of each region and the length of the primers are preferably chosen so that the Tm value of the primer in question and the corresponding template DNA lies between 50° C. and 70° C., and so the annealing temperature used in the PCR can be set at a relatively high value. The Tm value used here is a value that is calculated by the nearest neighbor base pair analysis. The primers can have the same Tm value.

PCR may be a preferred method of detection according to the present invention, and it can be carried out in accordance with normal PCR protocols, provided that the DNA obtained from the sample may be used as a template, and a specific primer set according to the invention. In particular, real-time PCR is employed in the method according to the invention (real time PCR assay, see the Examples), because this has given particularly good results.

The primer pair can be designed in such a way that the nucleotide sequence between the two regions (i.e., the region between the location where a first primer will bind under the PCR conditions and the location where a second primer will bind under the PCR conditions) can be replicated (amplification). This means that one primer can be a sense primer, and the other can be an anti-sense primer. Designing the nucleotide sequence of the primer pair can be based on nucleotide sequences of CT and NG, for instance, SEQ ID NO:1 and SEQ ID NO:6 as disclosed herein. SEQ ID NO:1 is one of the DNA strands of the double-stranded cryptic plasmid. This strand depicted here is the sense strand. The opposite strand, which is complementary to this sense strand, is the anti-sense strand. As for the amplification with the aid of, for example, PCR, the person skilled in the art will know that he should base the sequence of the primer of the primer pair to be used on the sequence described above, which corresponds to the sequence of the sense strand of the cryptic plasmid (the so-called “forward” primer). He will base the sequence of the other primer of the primer pair on the sequence of the anti-sense strand, called the “reverse” primer.

In the method of the invention, a “control DNA” is provided. A control DNA according to the invention is used to control the sample processing and/or DNA amplification. In the case where no signal is detected for CT as well as NG, it is important to be able to confirm that the test result for CT and NG is negative, and that an error has not been made, or any other cause that has led to a negative result. For example, sample DNA may have been lost during the preparation of the sample, or the sample may comprise compounds that can inhibit the PCR reaction. The control DNA serves as a control thereto, and in case CT and NG are negative, and the control DNA is detected, the test can be approved and it can be concluded the sample is negative for CT and NG. In contrast, if CT, NG as well as control DNA are all not detected, the test result is not approved, and the test has to be repeated. Usually, the control DNA comprises a sequence between the two primer binding sites on the DNA that would not interfere with the detection of the specific target sequence, for instance, artificial DNA sequences are inserted between the two primer binding sites on the control DNA.

Control DNA sequences are known in the art but these only serve as a control for a single amplification reaction. For instance, the Roche CT/NG AMPLICOR™ assay as described above uses a CT target as control. In contrast, the control DNA of the invention can be amplified with one of the primers for a first organism, such as Chlamydia trachomatis and one of the primers for a second organism, such as Neisseria gonorrhoeae. This principle can be extended to many more microorganisms. This concept is in particular advantageous, as according to the invention, the control DNA now serves as a control for both amplification reactions. Only when one specific primer of each primer pair for each molecular target is present the control DNA can be amplified. The control DNA is preferably double stranded, but may also be single stranded.

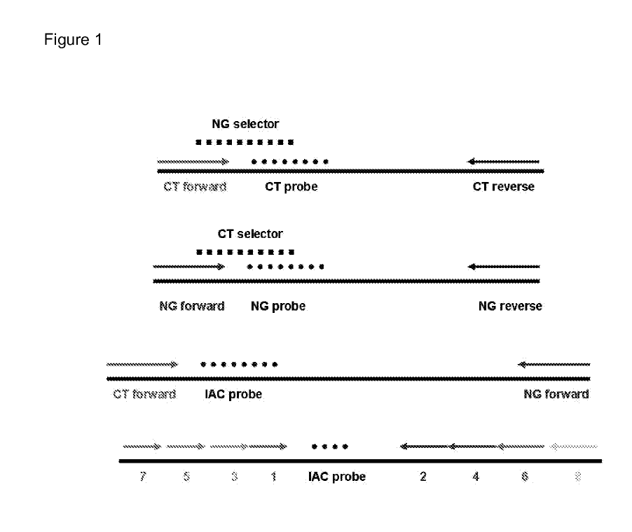

In between the primer binding sites, a sequence may be inserted, which does not result in a positive signal for the at least two molecular targets. Such an inserted sequence preferably is unrelated, and may be a naturally occurring sequencing, or may be an artificial sequence. For instance, CT is amplified with a CT forward primer A and CT reverse primer B, and NG is amplified with NG forward primer C and NG reverse primer D (see FIG. 1). The skilled person may design or select a control DNA that can be amplified with any of the following four combinations: primer A and C, A and D, B and C, and B and D. The control DNA thus comprises part of a NG sequence and part of a CT sequence to which the primers may bind. Such sequences may comprise the same primer binding sites as the corresponding CT and NG targets, but this is not mandatory, as long as one of the primers for NG and one of the primers for CT can hybridize with the control DNA and amplify it.

According to the invention, the DNA sample is preferably provided in a container. The DNA sample according to the invention may be obtained from an organism, such as a patient. For ease of handling and transportation, it can be contained in a container. The container comprising the DNA sample may be sent to a laboratory for testing and/or stored before testing. The control DNA may be added to the container or may already have been present before the DNA sample was contained in the container. The container comprising the control DNA and/or DNA sample may be further prepared or processed such that DNA amplification and thus detection of CT, NG or control DNA can be performed. Such preparation may be lysis of the DNA sample. For instance, a solution comprising guanidine isothiocyanaat may be added to the DNA sample such that the cells are lysed, thereby releasing the DNA into the solution. The DNA sample may be further processed in order to largely remove constituents other than the DNA from the DNA sample, as these other constituents may have PCR-inhibiting properties. For instance, any commercial DNA isolation kit may be used to prepare the DNA from a DNA sample. The DNA can be isolated resulting in a relatively pure DNA sample, or it may be in an unprocessed lysate prior to amplification. Whichever method is employed, it has been found to be, in particular, advantageous to add the control DNA prior to the preparation of the DNA sample. By adding the control DNA at an early stage, the control DNA not only serves as a control for the DNA amplification, but also as a control for the entire sample preparation/handling process. This is advantageous if during the preparation of the sample DNA the DNA would be lost for some reason, the test result would also be negative for the control DNA, as the control DNA would be lost as well.

The sequence length of the control DNA is larger as compared to the sequence length of the first and the second molecular target of the DNA sequence that can be amplified (see FIG. 1). For instance, in the case where the CT and NG amplified sequences are 100 nucleotides in length, the sequence length of the control DNA may be 101, more preferably 102, even more preferably 110, most preferably 120, or even larger. Also, the size of the control DNA is limited, as the larger it becomes, the more time it takes for efficient amplification; given the elongation rate of DNA polymerase and standard PCR reactions, the control DNA preferably is less than 3 kilobases, more preferably less than 1.5 kilobases, most preferably less than 500 bases, most preferred less than 200 bases in size. Preferably, the size of the control DNA is large enough that the DNA of a first molecular target, such as CT, or the DNA of a second molecular target, such as NG, is amplified during PCR, but it is not so large that it can be efficiently amplified. Such a larger sequence length of control DNA is suitable for use herein.

It is well known that in PCR reactions, the size of the DNA that is amplified is important. This is, in particular, important when multiple molecular targets are amplified. For instance, in the AMPLICOR™ CT/NG test as described above, the size of the control is designed for equivalent amplification. In general, DNA fragments that are smaller will amplify more efficiently as compared to larger DNA fragments. The CT/NG Internal Control was designed to be equivalent to ensure that the CT/NG Internal Control is always detected. According to the invention, it has been found that it is, in particular, advantageous to increase the size of the control DNA, as this increases the sensitivity of the DNA amplification. Indeed, as the larger control DNA has a disadvantage over the CT and/or NG DNA to be amplified, this may not lead to the detection of the control DNA when CT and/or NG is detected. Surprisingly, it has been found that this is not problematic, and even advantageous, as the sensitivity for detecting CT and/or NG increases. In case where the test result for control DNA is negative, this does not lead to the conclusion that the test per se has to be rejected, as the test result is positive for either CT and/or NG (i.e., in the case where the test result is negative for all, the test would not be approved). Hence, control DNA detection is not required to know whether or not the test has functioned appropriately, as detection of CT and/or NG detection is, in itself, a control for the test. In DNA samples that comprise very low amounts of CT and/or NG, standard internal controls such as NG/CT Internal Control may become a competitor during the amplification process, thereby disallowing the detection of minute amounts of CT and/or NG.

In a further embodiment, the control DNA is preferably part of plasmid DNA. More preferably, a microorganism, preferably a bacterium, comprises the control DNA. The control DNA may be part of plasmid DNA. The control DNA is preferably part of plasmid DNA as plasmid DNA is convenient to propagate and relatively easy and economical to prepare, but also because plasmid DNA serves as a better control as it may be more stable and is more equal in size as compared to the CT and NG DNA that serve as a template during DNA amplification. Furthermore, a microorganism, preferably a bacterium, comprises the control DNA, but other organisms are also envisaged depending on the molecular targets for which detection is monitored. As such, the control DNA serves as an even better control for the CT and NG microorganisms, as the control is also part of a microorganism. The control DNA may comprise the control DNA as part of plasmid DNA.

Alternatively, and most preferred, the control DNA is incorporated in the genome of the microorganism, this way, a single microorganism comprises a single copy of the control DNA in its genome, which advantageously more closely resembles the organisms for which it controls. Such a microorganism is preferably inactivated before it is used as an internal control, for example, through heat inactivation by heating at 80° C. for 30 minutes, however, other methods of inactivation are also envisaged. Importantly, the control DNA being part of a microorganism most closely resembles the CT and NG organisms that comprise DNA that is to be amplified and detected. Such a particular microorganism comprising control DNA in its genome is also referred to as an Internal Assay Control (IAC) in the example section below. For instance, in the case where lysis of a DNA sample would be ineffective and the control DNA would be merely added (i.e., not in a microorganism), the ineffective lysis would not result in DNA from the organisms that can be amplified and detected, however, the control DNA will be detected as it is not affected by the lysis, which will thus lead to false negative results. In contrast, in case of ineffective lysis, the lysis of a microorganism comprising control DNA would also be ineffective and thus would lead to a negative result for the control DNA as well as a negative result for CT and NG. As such, a natural microorganism comprising the control DNA serves as the best control according to the invention. Nevertheless, other ways of providing the control DNA are not excluded by the invention, as long as the control DNA can be amplified by one of the primers for CT and one of the primers for NG.

Good results may be obtained with the method of the invention when the primer pair for CT is designed on the basis of nucleotide sequences of the regions corresponding to the nucleotide numbers 3654 to 4320 and 4351 to 4448 of the nucleotide sequence of SEQ ID NO:1. More specifically, good results may be obtained when the primer pair for Chlamydia trachomatis is designed on the basis of the nucleotide sequences GGATTGACTCCGACAACGTATTC (SEQ ID NO:2) and TGCCCTTTCTAATGGCAATGAT (SEQ ID NO:3). More in particular, the primer pair used for Chlamydia trachomatis is 5′-GGATTGACTCCGACAACGTATTC-3′ (SEQ ID NO:4) and 5′-ATCATTGCCATTAGAAAGGGCA-3′ (SEQ ID NO:5).

Likewise, good results may be obtained when the primer pair for Neisseria gonorrhoeae is designed on the basis of nucleotide sequences of the regions corresponding to the nucleotide numbers 1-200 and 201-600 of the nucleotide sequence of SEQ ID NO:6. More specifically, good results may be obtained when the primer pair for Neisseria gonorrhoeae is designed on the basis of nucleotide sequences GTTGAAACACCGCCCGG (SEQ ID NO:7) and ATCTTTTTTTAACCGGTCAAACCG (SEQ ID NO:8). More in particular, the primer pair used for Neisseria gonorrhoeae is 5′-GTTGAAACACCGCCCGG-3′ (SEQ ID NO:9) and 5′-CGGTTTGACCGGTTAAAAAAAGAT-3′ (SEQ ID NO:10).

The person skilled in the art will understand that the term “designed on the basis of” means that, with the stipulations specified for the primer, such as the Tm value and the length of the primer, the primer is designed such that it can be complementary to the sequence in either the sense strand or the antisense strand of the nucleotide sequences according to the invention described herein. The design, therefore, starts with the sequence on which the primer has to bind, and with the stipulations and in the context of the present invention, the primer can be complementary to the sequence on which it has to bind. For example, the primer design is based on one of the DNA strands, for instance, the sequence of SEQ ID NO:1. One primer corresponds to a sequence from SEQ ID NO:1, while the other primer is complementary thereto, for instance, the primer 5′-GGATTGACTCCGACAACGTATTC-3′ (SEQ ID NO:4) corresponds to the sequence GGATTGACTCCGACAACGTATTC (SEQ ID NO:2), while the primer 5′-ATCATTGCCATTAGAAAGGGCA-3′ (SEQ ID NO:5) is complementary to TGCCCTTTCTAATGGCAATGAT (SEQ ID NO:3).

Accordingly, the control DNA preferably comprises nucleotide sequences corresponding to the nucleotide numbers 3654 to 4320 or 4351 to 4448 of the nucleotide sequence of SEQ ID NO:1 and nucleotide sequences corresponding to the nucleotide numbers 1-200 or 201-600 of the nucleotide sequence of SEQ ID NO:6, more preferably the control DNA comprises nucleotide sequences corresponding to SEQ ID NO:2 or SEQ ID NO:3 and SEQ ID NO:7 or SEQ ID NO:8.

The above preferred sequences have been found to be particularly useful for the current invention, as these were found to yield, in particular, good results.

Furthermore, according to the invention, the first molecular target, the second molecular target and/or the control DNA are monitored with one or more hybridization probes. The first and second molecular targets may be Chlamydia trachomatis and Neisseria gonorrhoeae. Amplified DNA can be detected using several standard techniques available to the person skilled in the art. For instance, a simple method would involve gel electrophoresis with ethidium bromide staining and a gel marker that would detect amplified DNA based on size and fluorescence. Alternatively, amplified DNA may also be detected with Southern blot with a (radioactively) labeled probe capable of hybridizing to amplified DNA. More advanced techniques, such as, for instance, real-time quantitative PCR (qPCR), are currently part of the standard practice of the skilled person and may also be employed. These latter techniques employ hybridization probes, and it is preferred that the CT, NG and/or control DNA is detected using hybridization probes, but other techniques are not excluded by the invention. With the term “hybridization probe” is meant a polynucleotide that is complementary to either the sense or the antisense strand of a DNA that is to be detected. Such complementarity may be perfect, that is, a G base pair with a C, and an A base pair with a T; however, it is also understood that occasional mismatches may be allowed, as long as the hybridization probe can bind with the amplified product and allow detection, and as long as the hybridization probe results in the specific detection, e.g., does not hybridize with primers and is selective between control DNA, NG and CT. Most preferably, the following probes are used: for NG, probe 1 for NG strain 1, 5′-CCCTTCAACATCAGTGAAA-3′ (SEQ ID NO:14) probe 2 for NG strain 2, 5′-CTTTGAACCATCAGTGAA A-3′ (SEQ ID NO:15) or probe 3 for both strain 1 and 2 of NG, 5′-ACCCGATATAATCCG-3′ (SEQ ID NO:16) may be used; for CT, 5′-ACACCGCTTTCTAAACCGCCTACACGTAA-3′ (SEQ ID NO:17), and for control DNA, 5′-TCTGGCGAAAGATTTGGCGGATGTGCATT (SEQ ID NO:18).

Furthermore, it has been found in the invention that occasionally for samples the signal for one of either CT or NG or any other molecular target of which detection may be monitored can be very strong, such that it is difficult to reliably determine the presence of the other species, NG or CT, respectively. This happens in a minority of DNA samples. Currently, the skilled person has no other option than to perform a separate assay in which one of the primer pairs that results in the strong signal that may need to be avoided, is excluded. This means that the skilled person needs to be able to combine different primer pairs for different molecular targets, as it is desirable to be able to exclude one or more primer pairs for particular molecular targets should these result in strong signals masking potential signals of other molecular targets. This means that the skilled person needs a multitude of different kits and/or components and the methods become laborious as all the components need to be provided and combined separately. Furthermore, in the case where either NG or CT provides for a strong signal, this has the disadvantage that the control DNA as described above may no longer be used. Thus, it is difficult to compare the test result from a separate assay for only CT or NG with the outcome of a combined monitoring of detection for both NG and CT.

According to the invention, for one of the primer pairs only a single primer is provided, the primer being involved in the amplification of the DNA of one of the molecular targets and the control DNA. Thus, for instance, one of the primers for CT or NG as described above is omitted, wherein the omitted primer is not involved in amplification of the control DNA, thereby preventing either CT or NG DNA amplification. When, for instance, CT provides for a very strong signal, using exactly the same processing of the DNA sample, e.g., sample preparation, DNA amplification and detection NG can be reliably detected. Advantageously, the same control DNA may be used, however, the disadvantage is that all the primers and/or probes need to be combined, which is highly laborious, and primers and/or probes need to be provided separately.

Alternatively, and most preferably, a selector nucleic acid is provided capable of preventing hybridization of one of the primers of one of the primer pairs and/or one of the hybridization probes with the DNA of the corresponding molecular target, the primer and/or probe being involved in the amplification and/or monitoring of the detection of one of the molecular targets. Thus, the selector nucleic acid does not prevent the hybridization of the primer for the first or second molecular target, which is also capable of amplifying the control DNA.

Likewise, a selector nucleic acid is provided capable of preventing hybridization of a primer for either Chlamydia trachomatis or Neisseria gonorrhoeae, wherein the primer can only be involved in the amplification of the Chlamydia trachomatis or Neisseria gonorrhoeae DNA. Such a selector nucleic acid does not prevent hybridization of a primer capable of amplifying control DNA as well as CT or NG DNA. Such a selector nucleic acid may, for instance, be capable of hybridizing to Chlamydia trachomatis DNA or Neisseria gonorrhoeae DNA at or around the hybridization site of the primer involved only in amplification of the control DNA and/or at or around the hybridization site of the probe, thereby preventing either Chlamydia trachomatis or Neisseria gonorrhoeae DNA amplification and/or hybridization of the probe to CT or NG DNA. Such a selector nucleic acid, in fact, may be similar to the primer or the probe and comprise largely the same sequence, except that the DNA polymerase is incapable of elongating the selector nucleic acid. Such a selector nucleic acid may, for instance, have its 3′-end phosphorylated, thereby preventing DNA elongation, as DNA polymerase requires the 3′-end to have a free-hydroxyl group. Other modifications are also envisaged, as long as DNA elongation is seriously hampered or prevented and hybridization of the primer prevented.

By providing an excess of such a selector nucleic acid, which may also have an increased affinity by the incorporation of modified nucleotides, such as LNAs, or by having an increased size as compared to the primer and/or probe, hybridization of the primer involved only in amplification of either NG or CT is prevented and thus the exponential amplification of either NG or CT is prevented. With “prevention of binding” is meant that the amount of primer that can bind to NG or CT DNA is severely reduced. The skilled person understands that, in principle, it may still be possible that some primers bind to CT or NG DNA in the presence of the selector, but that this does not lead to an amplified product. With a “selector nucleic acid” is thus meant a nucleic acid molecule that prevents the hybridization of the primer with either CT or NG that is only involved in CT or NG amplification (and, thus, not amplification of the control DNA). Thus, a selector nucleic acid according to the invention may strongly (and irreversibly) bind directly with the primer, thereby also preventing the hybridization of the primer with the CT or NG DNA present in the sample. As long as the selector nucleic acid is capable of preventing hybridization of the primer with CT or NG DNA, thereby preventing amplification, such selector is suitable for the invention. By preventing such hybridization of a primer with either CT or NG DNA, the strong signal masking possibly weaker signals may be detected, using exactly the same conditions and samples as provided in the combined CT and NG detection. This has the advantage that no additional kits or methods are required and that the results from both assays can be directly compared.

Particularly, a selector nucleic acid is provided for Chlamydia trachomatis having the sequence 5′-TCGGTTTGACCGGTTAAAAAAAGATTTTCACTGAT-3′ (SEQ ID NO:11) or for Neisseria gonorrhoeae having the sequence 5′-GGATTGACTCCRACAACGTATTCATTACGTGTAG-3′ (SEQ ID NO:12). These selectors are preferably phosphorylated at their 3′-end.

In another embodiment, a method is provided for combined monitoring of detection of at least two molecular targets, which method comprises providing a DNA sample, a primer pair for a first molecular target and a primer pair for a second molecular target and amplifying the DNA with the primer pairs, characterized in that a selector nucleic acid is provided capable of preventing hybridization of one of the primers of one of the primer pairs and/or one of the hybridization probes, with the DNA of the corresponding molecular target, thereby preventing amplification of one of the molecular targets. Thus, a selector may be provided in any method for monitoring of detection of at least two organisms, in case it is desirable to prevent the amplification and/or amplification of one of the organisms.

In further embodiments, the invention provides for a control DNA having a sequence according to SEQ ID NO:13, a plasmid DNA comprising a sequence according to SEQ ID NO:13, a microorganism, preferably a bacterium, most preferably Escherichia coli, comprising SEQ ID NO:13, and a selector nucleic acid according to SEQ ID NO:11 or SEQ ID NO:12. Instead of a control DNA having a sequence according to SEQ ID NO:13, SEQ ID NO:19 may also be used as control DNA herein or in any other embodiment of the invention wherein SEQ ID NO:13 is used.

Furthermore, in another aspect of the invention, kits are provided, wherein the kits are particularly suited to perform the methods for combined detection of at least two organisms, such as CT and NG as described below. In a first embodiment, a kit is provided for combined monitoring of detection of at least two molecular targets, wherein the kit comprises:
a primer pair for a first molecular target;
a primer pair for a second molecular target; and
a control DNA;

wherein the control DNA can be amplified with one of the primers from the primer pair for the first molecular target and one of the primers from the primer pair for the second molecular target.

The kit comprises, preferably in addition:
a hybridization probe for the first molecular target;
a hybridization probe for the second molecular target; and
a hybridization probe for the control DNA.

Furthermore, the kits above may further comprise a selector nucleic acid for the first molecular target and/or the second molecular target. Such a kit may be particularly suited to avoid a strong signal of one of the molecular targets that may mask the detection of other molecular targets.

Another kit provided by the invention is a kit for combined monitoring of at least two molecular targets wherein the kit comprises:
a primer pair for a first molecular target;
a primer pair for a second molecular target;
a selector nucleic acid for the first and/or second molecular target.

Furthermore, in another embodiment, a kit is provided comprising:
a container comprising a primer pair for a first molecular target;
a container comprising a primer pair for a second molecular target; and
a container comprising control DNA;

wherein the control DNA can be amplified with one of the primers from the primer pair for the first molecular target and one of the primers from the primer pair for the second molecular target.

In a first embodiment, the kit further comprises:
a container comprising the primer for both the first molecular target and the control DNA; and
a container comprising the primer for both the second molecular target and the control DNA.

In a second embodiment, the kit further comprises:
a container comprising a selector for the first molecular target; and
a container comprising a selector for the second molecular target.

In another embodiment of the invention, a kit is provided comprising:
a container comprising a primer pair for a first molecular target and a primer pair for a second molecular target; and
a container comprising control DNA;

wherein the control DNA can be amplified with one of the primers from the primer pair for the first molecular target and one of the primers from the primer pair for the second molecular target.

In a preferred embodiment, the container comprising the primer pairs comprises, in addition, the probes for the first molecular target, the second molecular target and/or control DNA. According to these embodiments, the kits may further comprise:
a container comprising a selector for the first molecular target; and
a container comprising a selector for the second molecular target.

In an alternative embodiment, a kit is provided for combined monitoring of at least two molecular targets wherein the kit comprises:
a container comprising a primer pair for a first molecular target;
a container comprising a primer pair for a second molecular target;
a container comprising a selector for the first molecular target; and
a container comprising a selector for the second molecular target;

wherein preferably the primer pairs for the first and second molecular target are combined in one container.

The kits in which all of the primer pairs and/or probes are combined in a single container are particularly suited for the invention, as this is most convenient for monitoring of detection of at least two molecular targets. Furthermore, by providing, in addition, selectors for a molecular target, preferably for each molecular target, the kit prevents (or seriously hampers the amplification of a particular target (or even multiple targets) that would be masking the monitoring of detection of the other molecular targets in the assay.

In a preferred embodiment, in all of the kits above, the first molecular target is Chlamydia trachomatis and the second molecular target is Neisseria gonorrhoeae, wherein the kit comprises primer pairs as described above, CT primer pairs are preferably designed on the basis of nucleotide sequences of the regions corresponding to the nucleotide numbers 3654 to 4320 and 4351 to 4448 of the nucleotide sequence of SEQ ID NO:1, more preferably on the basis of the nucleotide sequences GGATTGACTCCGACAACGTATTC (SEQ ID NO:2) and TGCCCTTTCTAATGGCAATGAT (SEQ ID NO:3), and most preferably the primer pair for CT is 5′-GGATTGACTCCGACAACGTATTC-3′ (SEQ ID NO:4) and 5′-ATCATTGCCATTAGAAAGGGCA-3′ (SEQ ID NO:5), NG primer pairs are preferably designed on the basis of nucleotide sequences of the regions corresponding to the nucleotide numbers 1-200 and 201-640 of the nucleotide sequence of SEQ ID NO:6, more preferably on the basis of nucleotide sequences GTTGAAACACCGCCCGG (SEQ ID NO:7) and ATCTTTTTTTAACCGGTCAAACCG (SEQ ID NO:8), and most preferred is the primer pair 5′-GTTGAAACACCGCCCGG-3′ (SEQ ID NO:9) and 5′-CGGTTTGACCGGTTAAAAAAAGAT-3′ (SEQ ID NO:10). Preferably, the control DNA for CT and NG is as described herein, and on the basis of the sequences of CT and NG above and, most preferably, has a sequence according to SEQ ID NO:13. Furthermore, a selector is preferably provided for CT and NG as described herein, preferably having a sequence according to SEQ ID NO:11 or SEQ ID NO:12, respectively. Finally, hybridization probes are preferably provided as described herein for CT (SEQ ID NO:17), NG (SEQ ID NO:14, SEQ ID NO:15 and/or SEQ ID NO:16), and/or control DNA (SEQ ID NO:18).

The invention is explained in more detail with the aid of the following examples and drawings.

### Example 1

### Materials:

The following materials were provided: H₂O, 10× Invitrogen buffer, Bovine Serum Albumin (BSA, 1.25%), 100 mM dNTP, 1 M MgCl₂, ROX, NaH₃ (5%), Taq DNA polymerase. The following primers were provided:

Fct (1000 μM)
(SEQ ID NO: 2)
5′-GGA TTG ACT CCG ACA ACG TAT TC -3′,
Rct (1000 μM)
(SEQ ID NO: 5)
5′-ATC ATT GCC ATT AGA AAG GGC A-3′,
Fgo (1000 μM)
(SEQ ID NO: 7)
5′-GTT GAA ACA CCG CCC GG-3′,
Rgo (1000 μM)
(SEQ ID NO: 10)
5′-CGG TTT GAC CGG TTA AAA AAA GAT-3′.

The following probes were provided:

pCT-FAM-BHQ (100 μM)
(SEQ ID NO: 17)
5′-^{FAM} ACA CCG CTT TCT AAA CCG
CCT ACA CGT AA^{BHQ1}-3′,
pgo1MGB-VIC (100 μM)
(SEQ ID NO: 14)
5′-^{VIC} CCC TTC AAC ATC AGT GAA
A ^{MGB}-3′,
po2MGB- VIC (100 μM)
(SEQ ID NO: 15)
5′-^{VIC} CTT TGA ACC ATC AGT GAA
A ^{MGB}-3′,
Pgo3MGB-VIC (100 μM)
(SEQ ID NO: 16)
5′-^{VIC} ACC CGA TAT AAT CCG ^{MGB}-3′,
IAC-Cy5 (100 μM)
(SEQ ID NO: 18)
5′-^{Cy5} TCT GGC GAA AGA TTT GGC GGA
TGT GCA TT^{BHQ2}-3′.

PCR amplification can be carried out with any real-time PCR apparatus able to detect the fluorophores: FAM, VIC, ROX & Cy5. The following selectors were provided:

CT selector (1000 μM)
(SEQ ID NO: 11)
5′-TCG GTT TGA CCG GTT AAA AAA
AGA TTT TCA CTG AT-3′,
NG selector (1000 μM)
(SEQ ID NO: 12)
5′-GGA TTG ACT CCR ACA ACG TAT
TCA TTA CGT GTA G-3′,

the selectors were phosphorylated at the 3′-end. Primers, probes and selectors were dissolved in H₂O. An internal control was also provided (IAC, internal assay control), an inactivated E. coli modified with a genomic DNA fragment containing primer binding sites identical to the C. trachomatis and N. gonorrhoeae sequences, but with a different intermediate probe sequence.

A master mix was prepared, comprising the four primers (Fct, Rct, Fgo, Rgo), three primers for CT, NG and IAC, and all components for PCR amplification were also incorporated in the master mix. Separate tubes were provided with the internal control (IAC), a CT Positive Control of CT DNA at 4 IFU/10 μl, an NG Positive Control Positive of NG DNA at 100 CFU/10 μl, a negative control, a CT selector for use with high NG positive samples for the detection of a weak positive CT-co-infection and an NG Selector for use with high CT positive samples for the detection of a weak positive NG-co-infection.

### Methods:

DNA sample: example of a swab specimen

1. Collect endocervical and urethral swab specimens and store them in 2-5 ml of 2SP transport medium.

2. Keep the swabs in the transport medium. Refrigerate (2° C.-8° C.) or freeze swab specimens that will not be processed immediately. Specimens can be stored for 7 days at 2° C.-8° C.

DNA sample: example of a urine specimen

1. Collect 10 to 30 ml of first catch urine into a clean polypropylene container without any preservatives.

2. Follow the laboratory's collection and transport procedures. Refrigerate (2° C.-8° C.) urine specimens that will not be processed immediately. Specimens can be stored for 7 days at this temperature.

DNA Isolated specimen:

These materials and methods are to be used with Nucleic Acid extracted samples by means of BioMerieux NUCLISENS® EASYMAG™. Please follow the manufacturer's instructions for a description of the system features, isolation protocols and operational guidelines.

Swabs: 200 μl vortexed specimen+5 μl resuspended IAC+2 ml EASYMAG™ lysis buffer and elution in 60 ml of which 10 μl is used in the CT/NG PCR.

Urine: 500 μl vortexed urine+5 μl resuspended IAC+2 ml EASYMAG™ lysis and elution in 60 μl of which 10 μl is used in the CT/NG PCR

### Test Procedure

1. Prepare the required number of reaction tubes or wells for the number of specimens to be measured, plus two tubes for the positive controls, one tube for the negative isolation control and one tube for the negative control.

2. Add 15 μl of the master mix to each reaction to be measured.

3. Vortex and spin down all DNA extracts. Carefully open specimen containers one by one and avoid contamination of gloves and pipette. Using a new aerosol barrier tip for each extract, add 10 μl DNA to the reaction tube/well containing the master mix. Replace gloves if suspect of contamination.

4. Using a new aerosol barrier tip, add 10 μl of each positive control Positive Control to the designated reaction tubes/wells containing the master mix. Carefully open the container and avoid contamination of gloves and pipette. Replace gloves if suspect of contamination.

5. Using a new aerosol barrier tip, add 10 μl of the negative control Negative Control to the designated reaction tube/well containing the master mix.

6. Close the reaction tubes or seal the plate, spin down and move the plate to the Amplification Area.

7. Load the reaction tubes/plate into the ABI PRISM® 7500 SDS. Program the PCR System with following settings:

| | |
|---|---|
| Fixed threshold: | 0.01 |
| Activation polymerase: | 30 seconds 95° C. |
| Number of cycles: | 40 cycles |
| Denaturation: | 3 seconds 95° C. |
| Annealing, extension and exonuclease activity: | 30 seconds 60° C. |
| Manual baseline settings | |

### Use of Selectors

In the event of a positive signal with either CT or NG, the PCR must be repeated with 1 μl of either of the selectors and 9 μl of isolated DNA; for CT positive samples, use NG selector; for NG positive samples, use CT selector. Amplification conditions are as described above.

By adding the selector, an IAC signal should appear (if no IAC signal appears, the PCR is inhibited). A weak signal for the initial positive target may still be present. In the event of a double infection, a signal for the other target will be visible.

### Example 2

The NG/CT test using methods and materials as described in Example 1 was compared with the test as provided by Roche (AMPLICOR™ CT/NG kit).

### Protocol

All DNA samples were isolated with Hamilton Starlight DNA isolation apparatus with the use of MagNA Pure LC DNA Isolation Kit—Large Volume. Briefly, 200 μl of the sample was isolated with elution in 60 μl. 10 μl of the isolated DNA was used in PCR with 15 μl of PCR mix and subsequently amplified in an ABI Prism SDS 7500. FAST Amplification protocol with 30 seconds denaturation/activation at 95° C. followed by 45 cycles of 95° C. for 3 seconds and 60° C. for 30 seconds. All samples were analyzed according to test instructions. A selection of inhibited samples were retested, with the use of an EASYMAG™ DNA extraction followed by amplification as described earlier. All extra positive samples were retested by repeating the amplification with the Hamilton isolated DNA as well as freshly isolated DNA with EASYMAG™ technology.

### DNA Sample List

TABLE 1
DNA sample list for CT/NG test.

| material | Total | Male | Female |
|---|---|---|---|
| Rectal | 603 | 343 | 260 |
| Urine | 1461 | 1289 | 172 |
| Endovaginal | 1772 | 1(*) | 1771 |
| Oropharyngeal | 200 | 112 | 88 |
| Total | 4036 | 1745 | 2291 |

(*)Transsexual

Table 1 describes the total number of samples included in the study and from which DNA was isolated with the Hamilton. Four different sample types were used in this study: Rectal, urine, endovaginal and oropharyngeal samples. The distribution of these samples types between males and females are shown above: 57% of the samples are from women, and 43% of the samples are from men.

TABLE 2
Total number of each sample type divided by gender

| Material | Total | Male | Female |
|---|---|---|---|
| Rectal | 603 | 343 | 260 |
| Urine | 1461 | 1289 | 172 |
| Endovaginal | 1772 | 1 | 1771 |
| Oropharyngeal | 200 | 112 | 88 |

### Results

TABLE 3
Cross-tabulation of the Roche and NG/CT test results for CT.

| NG/CT test CT | Total | Roche Neg | Inhibition | Pos |
|---|---|---|---|---|
| Repeat | 180 | 169 | 2 | 9 |
| Neg | 3016 | 2965 | 34 | 17 |
| New isolation | 518 | 500 | 4 | 14 |
| Pos | 319 | 16 | 3 | 300 |

Table 3 describes the results for the comparison of the Roche with the NG/CT test for Chlamydia trachomatis. Total numbers are shown, those samples positive, negative and those with inhibition. In addition, we showed the number of the samples marked for new isolations or repeat TaqMan analyses.

The NG/CT test was better when compared to the Roche test.

### Example 3

Different strains of NG may be tested using the same set of primers as described in Examples 1 and 2.

A first strain may be detected using probe 1; a second strain may be detected using probe 2; both strains may be detected using probe 3:

| (SEQ ID NO: 14) | |
|---|---|
| Probe 1 | ^{VIC}-CCC TTC AAC ATC AGT GAA A-^{MGB} |
| (SEQ ID NO: 15) | |
| Probe 2 | ^{VIC}-CTT TGA ACC ATC AGT GAA A-^{MGB} |
| (SEQ ID NO: 16) | |
| Probe 3 | ^{VIC}-ACC CGA TAT AAT CCG-^{MGB} |
| (SEQ ID NO: 7) | |
| Forward Primer | GTT GAA ACA CCG CCC GG |
| (SEQ ID NO: 10) | |
| Reverse primer | CGG TTT GAC CGG TTA AAA AAA GAT |
| | Neisseria gonorrhoeae strain 1 |
| 1 | TGTGTTGAAACACCGCCCGG AACCCGATATAATCCGCCCT TCAACATCAGTGAAAATCTT |
| | ACACAACTTTGTGGCGGGCC TTGGGCTATATTACGGGGGA AGTTGTAGTCACTTTTAGAA |
| 61 | TTTTTAACCGGTCAAACCGA ATAA |
| | AAAAATTGGCCAGTTTGGCT TATT |
| | Neisseria gonorrhoeae strain 2 |
| 1 | TGTGTTGAAACACCGCCCGG AACCCGATATAATCCGCCTT TGAACCATCAGTGAAAATCT |
| | ACACAACTTTGTGGCGGGCC TTGGGCTATATTAGGCGGAA ACTTGGTAGTCACTTTTAGA |
| 61 | TTTTTTAACCGGTCAAACCG AATAA |
| | AAAAAATTGGCCAGTTTGGC TTAT |

1. A method for combined monitoring of detection of at least two molecular targets, which method comprises:
providing a DNA sample, a control DNA, a primer pair for a first molecular target, and a primer pair for a second molecular target; and
amplifying the DNA with the primer pairs;
wherein said control DNA is able to be amplified with one of the primers for the first molecular target and one of the primers for the second molecular target and wherein a microorganism comprises the control DNA.

2. The method according to claim 1, wherein the first molecular target is Chlamydia trachomatis and the second molecular target is Neisseria gonorrhoeae.

3. The method according to claim 1, wherein the DNA sample is provided in a container.

4. The method according to claim 1, wherein the control DNA is provided in a container.

5. The method according to claim 1, wherein the sequence length of the control DNA is larger than the sequence length of the first molecular target and the second molecular target of the DNA sequence that can be amplified.

6. The method according to claim 1, wherein a plasmid comprises the control DNA.

7. The method according to claim 1, wherein the microorganism is a bacterium.

8. The method according to claim 2, wherein the primer pair for Chlamydia trachomatis is designed on the basis of nucleotide sequences of the regions corresponding to the nucleotide numbers 3654 to 4320 and 4351 to 4448 of the nucleotide sequence of SEQ ID NO:1.

9. The method according to claim 2, wherein the primer pair for Chlamydia trachomatis is designed on the basis of the nucleotide sequences GGATTGACTCCGACAACGTATTC (SEQ ID NO:2) and TGCCCTTTCTAATGGCAATGAT (SEQ ID NO:3).

10. The method according to claim 2, wherein the primer pair for Chlamydia trachomatis is 5′-GGATTGACTCCGACAACGTATTC-3′ (SEQ ID NO:4) and 5′ ATCATTGCCATTAGAAAGGGCA-3′ (SEQ ID NO:5).

11. The method according to claim 2, wherein the primer pair for Neisseria gonorrhoeae is designed on the basis of nucleotide sequences of the regions corresponding to the nucleotide numbers 1-200 and 201-640 of the nucleotide sequence of SEQ ID NO:6.

12. The method according to claim 2, wherein the primer pair for Neisseria gonorrhoeae is designed on the basis of nucleotide sequences GTTGAAACACCGCCCGG (SEQ ID NO:7) and ATCTTTTTTTAACCGGTCAAACCG (SEQ ID NO:8).

13. The method according to claim 2, wherein the primer pair for Neisseria gonorrhoeae have the following sequences 5′-GTTGAAACACCGCCCGG-3′ (SEQ ID No. 9 NO:9) and 5′-CGGTTTGACCGGTTAAAAAAAGAT-3′ (SEQ ID NO:10).

14. The method according to claim 2, wherein the control DNA is designed on the basis of the nucleotide numbers 3654 to 4320 or 4351 to 4448 of the nucleotide sequence of SEQ ID NO:1 and nucleotide sequences corresponding to the nucleotide numbers 1-200 and 201-640 of the nucleotide sequence of SEQ ID NO:6.

15. The method according to claim 2, wherein the control DNA comprises a nucleotide sequence corresponding to SEQ ID NO:2 or SEQ ID NO:3 and a nucleotide sequence corresponding to SEQ ID NO:7 or SEQ ID NO:8.

16. The method according to any of the preceding claim 1, wherein the first molecular target, the second molecular target and/or the control DNA are monitored with one or more hybridization probes.

17. The method according to claim 16, wherein the probe for Chlamydia trachomatis is SEQ ID NO:17, the probe for Neisseria gonorrhoeae is selected from the group consisting of SEQ ID NO:14, SEQ ID NO:15, and SEQ ID NO:16, and/or the probe for the control DNA is SEQ ID NO:18.

18. The method according to any of the preceding claim 1, wherein of one of the primer pairs only a single primer is provided, said primer being involved in the amplification of one of the molecular targets and the control DNA.

19. The method according to claim 1, wherein a selector nucleic acid is provided able to prevent hybridization of one of the primers of one of the primer pairs and/or one of the hybridization probes, with the DNA of the corresponding molecular target, the primer and/or probe being involved in the amplification and/or monitoring of detection of one of the molecular targets.

20. The method according to claim 19, wherein a selector nucleic acid is provided for Chlamydia trachomatis having the sequence 5′-TCGGTTTGACCGGTTAAAAAAAGATTTTCACTGAT-3′ (SEQ ID NO:11) and/or for Neisseria gonorrhoeae having the sequence 5′-GGATTGACTCCRACAACGTATTCATTACGTGTAG-3′ (SEQ ID NO:12).

21. A method for combined monitoring of detection of at least two molecular targets, which method comprises:
providing a DNA sample, a primer pair for a first molecular target and a primer pair for a second molecular target; and
amplifying the DNA with the primer pairs;
wherein a selector nucleic acid is provided able to prevent hybridization of one of the primers of one of the primer pairs and/or one of the hybridization probes, with the DNA of the corresponding molecular target, thereby preventing amplification of one of the molecular targets.

22. A control DNA comprising SEQ ID. NO:13.

23. A plasmid DNA to comprising SEQ ID NO:13.

24. A microorganism comprising the control DNA of claim 21.

25. A selector nucleic acid according to SEQ ID NO:11 or SEQ ID NO:12.

26. A kit for combined monitoring of detection of at least two molecular targets, wherein the kit comprises:
a primer pair for a first molecular target;
a primer pair for a second molecular target; and
a control DNA;
wherein said control DNA can be amplified with one of the primers from the primer pair for the first molecular target and one of the primers from the primer pair for the second molecular target.

27. The kit according to claim 26, wherein the kit further comprises:
a hybridization probe for the first molecular target;
a hybridization probe for the second molecular target; and
a hybridization probe for the control DNA.

28. The kit according to claim 26, wherein the kit further comprises a selector nucleic acid for the first molecular target and/or the second molecular target.

29. A kit for combined monitoring of at least two molecular targets wherein the kit comprises:
a primer pair for a first molecular target;
a primer pair for a second molecular target; and
a selector nucleic acid for the first and/or second molecular target.

30. A kit comprising:
a container comprising a primer pair for a first molecular target;
a container comprising a primer pair for a second molecular target; and
a container comprising control DNA;
wherein said control DNA can be amplified with one of the primers from the primer pair for the first molecular target and one of the primers from the primer pair for the second molecular target.

31. The kit of claim 30, further comprising:
a container comprising the primer for both the first molecular target and the control DNA; and
a container comprising the primer for both the second molecular target and the control DNA.

32. A kit comprising:
a container comprising a primer pair for a first molecular target and a primer pair for a second molecular target; and
a container comprising control DNA;
wherein said control DNA can be amplified with one of the primers from the primer pair for the first molecular target and one of the primers from the primer pair for the second molecular target.

33. The kit of claim 32, wherein the container comprising the primer pairs comprises in addition the probes for the first molecular target, the second molecular target and/or control DNA.

34. The kit of claim 30, further comprising:
a container comprising a selector for the first molecular target; and
a container comprising a selector for the second molecular target.

35. A kit for combined monitoring of at least two molecular targets, wherein the kit comprises:
a container comprising a primer pair for a first molecular target;
a container comprising a primer pair for a second molecular target;
a container comprising a selector for the first molecular target; and
a container comprising a selector for the second molecular target, target;
wherein preferably the primer pairs for the first and second molecular target targets are combined in one container.

36. The kit of claim 26, wherein the first molecular target is Chlamydia trachomatis and the second molecular target is Neisseria gonorrhoeae.

37. The kit of claim 36, wherein the sequence length of the control DNA is larger than the sequence length of the first molecular target and the second molecular target of the DNA sequence that can be amplified.

38. The kit of claim 36, further comprising a selector nucleic acid.

39. The kit of claim 36, wherein:
a probe comprises SEQ ID NO:17;
a probe comprises a molecule selected from the group consisting of SEQ ID NO:14, SEQ ID NO:15, and SEQ ID NO:16; and/or
a probe comprises SEQ ID NO:18.

40. A method for combined monitoring of detection of at least two molecular targets, the method comprising:
providing a DNA sample, a control DNA, a primer pair for a first molecular target, and a primer pair for a second molecular target; and
amplifying the DNA with the primer pairs;
wherein the control DNA is able to be amplified with one primer for the first molecular target and one primer for the second molecular target.
